(19) United States
(12) Patent Application Publication
(10) Pub. No.: US 2015/0166615 A1
(43) Pub. Date: Jun. 18, 2015

(54) Method and uses for Bombyx mori silk fibroin heavy chain mutation sequence and mutant

(71) Applicant: Southwest University, Chongqing (CN)

(72) Inventors: Qingyou Xia, Chongqing (CN); Sanyuan Ma, Chongqing (CN); Hanfu Xu, Chongqing (CN); Daojun Cheng, Chongqing (CN); Ying Lin, Chongqing (CN); Ping Zhao, Chongqing (CN); Zhonghuai Xiang, Chongqing (CN)

(21) Appl. No.: 14/348,898

(22) PCT Filed: Oct. 19, 2012
(86) PCT No.: PCT/CN2012/083175

§ 371 Date: Apr. 1, 2014

(30) Foreign Application Priority Data
Oct. 20, 2011 (CN) 201110319637.3

(51) Int. Cl. C07K 14/435

(52) U.S. Cl. CPC C07K 14/43586

(57) ABSTRACT

Provided is a method for Bombyx mori silk fibroin heavy chain mutation including causing the mRNA of a coded zinc-finger nucleases sequence to act upon a target point such as positions 1325-1362 of Bombyx mori silk fibroin heavy chain gene as expressed in SEQ ID NO:1 so as to obtain a series of Bombyx mori silk fibroin heavy chain mutated genes. Also provided is a mutation sequence prepared according to the method. Also provided is the use of the mutation sequence in the preparation of sericin and foreign proteins. Also provided is a Bombyx mori mutant including the described mutation sequence as a novel Bombyx mori salivary glands bioreactor, the posterior salivary glands of the mutants being severely degraded and the cocoon layer thereof including sericin synthesized and excreted by the middle salivary glands.

### BACKGROUND OF THE INVENTION

1. Field of the Invention

The invention relates to silkworm breeding and genomic engineering, involving based fibroin heavy chain gene mutants of different existing silkworm varieties and preparation methods, and use of the mutant sequence.

2. Description of Related Art

Silkworms are well known for its powerful ability to secrete silk for a long time. It makes an important contribution to breeding and domestication for thousands of year. It still plays a big role in countries like China and India. But for a long time breeding silkworm, cocoon varieties are based on volume, ratio of cocoon unwinding and improved disease resistance as breeding objectives, while ignoring the silk strength, elongation, dyeing performance, compatibility and other important skin indicators. It is also difficult for it to be used as high-strength fiber for applications in medical, military and other high-ends. This is why it is only used as a single textile material. Thus, how to fast breed silkworms which can produce cocoons having features such as quality fiber, sufficient strength, high elongation, being easy to die, and high affinity is very important.

After several years of silkworm breeding, although resources are more abundant species, but on silk strength, elongation, dyeing properties, skin affinity and other characteristics, there are almost no differences between the varieties. Thus, traditional breeding methods are not desired. With new methods being fully implemented, silkworm genome project, molecular marker-assisted breeding, transgenic breeding of modern agriculture gradually adds silkworm varieties to improved molecules field. Silk fibroin protein is comprised of heavy chain fibroin protein, light chain fibroin protein, P25 protein, and the outermost sericin protein in which the heavy chain fibroin protein determining the performance of silk is composed of highly repetitive sequences of about 390 kDa protein. Fibroin heavy chain protein has been highly expressed, highly repetitive sequences and molecular weight and thus it is difficult of studying and transforming it by using conventional transgenic technologies.

In addition, a silkworm having weight of about 6 g eating mulberry about 25 g, will be able to spit 0.5 g protein composed of pure protein. This is not only the breathtaking step of biology but also has many envisions. Further, it has the advantages of silkworm glands-translational modification of proteins in higher organisms processing capacity, low cost, mass production, and safety to human and animal. Thus, it meets a new generation of bioreactor requirements. There is a huge potential for its development and application. Research in this area has become an important part of expansion for non-traditional silk industry which evolves from traditional silk industry. Both in basic research and in drug development and in the field of cosmetics, extracts or purified protein expression plays a very important role. But the commercialization of pure protein bizarre high price (1 g most commonly used green fluorescent protein price is about 5 million RMB) has plagued a number of researchers. Lack of large scale extraction of protein limits the development of protein research and products. Cost of raising a silkworm is less than a dime. But it can spit silk protein 0.5 g. If genomic engineering can make silkworm spit 0.5 g of pure protein, which for the silk industry and even bio-industry, it will have a revolutionary role in silkworm raising promotion. It is also this strange biological phenomena and prospects attracts many biologists since the beginning of molecular biology and long term commitment to regulating the silkworm bioreactor developing the silk protein. However, to achieve this seemingly simple but beautiful dream is not easy. Despite the adoption of exploration and research for decades, the regulatory mechanism of the silk protein gene expression is not quite clear. Salivary glands bioreactor developed road is full of twists and turns out to have little success. Although the researchers used the silk protein gene promoter has seen success in different parts of the salivary glands expression of green fluorescent protein, red fluorescent protein, collagen, human basic fibroblast growth factor, interferon cat phytase, human serum albumin, mouse monoclonal antibody, spider dragline silk protein and brain-derived neurotrophic factor protein has significant research and application values. But now the salivary glands expression of exogenous gene transfer protein activity can only reach a maximum of 8.0% of the total weight of the cocoon. Expression level is too low. Cost of obtaining a small amount of protein from the purifying cocoon layer process is too high. Thus, scholars and businessmen who try to further develop the salivary glands bioreactor are very frustrated.

Whether for improvements of protein from domesticated silkworms or the development of practical, efficient salivary glands bioreactor, it is important to find an effective transformation of the silk protein gene designated method or rapid breeding silk protein mutant resources. However, although scholars in terms of silkworm gene targeting have done exploration, there is no available silkworm genome directional transformation technology.

Zinc-finger nucleases are an artificial restriction enzyme designed by the DNA sequences responsible for specific recognition domain and a zinc-finger protein. Enzyme is responsible for cleaving a nucleic acid consisting of the DNA domain. Zinc-finger nucleases can cause double-strand breaks in a complex genome of specific sites, thereby stimulating the body's DNA repair system: homologous end joining such as Non-homologous End Joining (NHEJ) and homologous recombination (HR). NHEJ is a special kind of DNA double-strand break repair mechanisms. DNA ends its fast, efficient forced break in the case of DNA homologous sequences that do not exist together. This is an incorrect repair mechanism. It is easy to introduce a small number of bases at the lack of repair, insertion or mutation. Fruit designing two pairs of zinc-finger nucleases in the same large DNA fragments may also cause large fragment between the two recognition sites missing. In the presence of faults exogenous DNA having a terminal case of the same cohesive ends, NHEJ can also insert specific small fragments. HR body is a common DNA repair mechanism. Its principles since the 1980s have been used widely in the knockout. By artificially designed for various types of plasmid, with the zinc-finger nucleases mediated double-strand breaks can be efficiently performed on the fixed target gene replacement, repair, delete, and insert an arbitrary operation. At present, this technology has been widely used in animal and plant genes knocked addition and transformation.

### SUMMARY OF THE INVENTION

It is therefore one object of the invention to provide a mutation of a heavy chain of fibroin of silk emitted by Bombyx mori comprising genes 1325 to 1362 of a heavy chain of fibroin of silk emitted by Bombyx mori mutated as mutated genes of a target of identified zinc-finger nucleases.

Preferably, the mutated genes of a target of identified zinc-finger nucleases are any ones of SEQ. ID NO: 2-118.

Preferably, the mutated genes of a target of identified zinc-finger nucleases are any ones of SEQ. ID NO: 2-14.

Preferably, the mutated genes of a target of identified zinc-finger nucleases are SEQ. ID NO: 11.

Preferably the mutated genes of a target of identified zinc-finger nucleases are described in the preparation of endogenous proteins.

Preferably, the mutated genes of a target of identified zinc-finger nucleases are described in the preparation of exogenous proteins.

It is another object of the invention to provide a method of mutation of a heavy chain of fibroin of silk emitted by Bombyx mori, comprising the steps of inserting a zinc-finger nucleases sequence containing SEQ. ID NO: 119 and SEQ. ID NO: 120 into a prokaryotic expression vector of a promoter containing T7: ′-taatacgactcactataggg-3′ or SP6: 5′-atttaggtgacactatag-3 to create a recombinant vector; transcribing in vitro of the recombinant vector to obtain an mRNA of coded zinc-finger nucleases; taking the mRNA of coded zinc-finger nucleases as genes 1325 to 1362 of a heavy chain of fibroin of silk emitted by Bombyx mori mutated as mutated genes of a target of identified zinc-finger nucleases and indicated by SEQ. ID NO: 1; and obtaining a mutation of a heavy chain of fibroin of silk emitted by Bombyx mori.

Preferably, the mutated genes of a target of identified zinc-finger nucleases are described in the preparation of sericin.

By utilizing the invention the following advantages are obtained:

The invention utilizes a newly developed zinc-finger nucleases technology, and efficient for fibroin heavy chain gene is knocked out for the first time received a partial deletion of genes included in the N-terminal non-repetitive region of fibroin heavy chain, part of the mutation or Small fragments into a series of strains of mutant silkmoths. Further, posterior salivary glands fibroin provided by heavy chain gene mutants is severely degraded, its cocoon layer contains only the middle salivary glands sericin synthesis and secretion protein. If the use of these mutant strains in the posterior salivary glands of transgenic expression of foreign proteins (such as spider silk protein biological activity), an exogenous protein in salivary glands, and the expression level will greatly improve the purity of the home, which is salivary glands bioreactor developed to provide a new and useful genomic material. Since the invention provides a mutant strain of cocoon sericin layer only, pure sericin has now been widely used in cosmetics. The invention can be used for mass production of sericin and thus provides a new source therefore.

The above and other objects, features and advantages of the invention will become apparent from the following detailed description taken with the accompanying drawings.

### BRIEF DESCRIPTION OF THE DRAWINGS

FIG. 1 shows a schematic structure of fibroin heavy chain gene, wherein the block exon gray solid line represents a regulatory region or intron sequences relative to the number indicates the position of the transcription initiation site, the underlined portion of the signal nucleotide or amino acid sequence of the peptide sequence, signal peptide sequence arrows cleavage site;

FIG. 2 shows a partial sequence of fibroin heavy chain gene SNP distributed in forty silkworm strains, in which the top of the box and the solid line represents the area of the location and structure of fibroin heavy chain gene analysis, the following forty one solid lines shows 30 nucleotide sequence of Bombyx mori silkworm strains and 11 field strains, the solid line indicates the corresponding rhombic block line with respect to the variation of the reference sequence number indicating SNP position relative to the transcription starting point locus starting position, and the solid line on the right is numbered number of different strains of the silkworm;

FIG. 3 shows the nucleotide sequence of the mutant fibroin heavy chain mutation, in which the corresponding number on the left side of the individual numbers, >Ref represents a sequence of wild strains of creating a big sequence, as shown in the box for zinc-finger nucleases recognition sites for the bold mutated nucleotide bases, - means deletion and the underline represents added nucleotide bases;

FIG. 4 shows the wild and mutant Bombyx mori silkworm salivary glands anatomy, which left for the wild strain and creating a big five age six days of salivary glands, the right to age six days of salivary glands of five mutant silkmoths;

FIG. 5 shows the mutant silkmoth cocoons observation chart, which ranked as the mutant strain on the N4 cocoon, the lower row of mutant silkmoth cocoons;

FIG. 6 shows the wild silkmoth cocoons observation chart, which ranked as the wild strain on the N4 cocoon, the lower row of wild silkmoth cocoons;

FIG. 7 shows the statistical analysis of biological mutant and wild silkmoth chrysalis silkworm cocoons and weight;

FIG. 8 shows a wild silkmoth, respectively, heterozygous and homozygous mutant mori silkworm mutant receptor transgenic silkworm cocoon protein analyzes, Transgenic-1, Transgenic-1 and Non-transgenic denote two transgenic lines and a transgenic line, +/+, +/− and −/−, respectively, of wild silkmoth, heterozygous mutant silkmoth and pure and mutant Bombyx mori, the bottom row numbers indicate lane numbers, arrows as exogenous GFP fusion protein specific bands;

FIG. 9 shows a table of recombinant vector and a specific enzyme reaction system;

FIG. 10 shows a table of reaction system;

FIG. 11 shows a table of another reaction system; and

FIG. 12 shows a table of microinjection and mutation screening results.

In biology, chromosomes are present in pairs, i.e., there are two same gene, a mutant of any one, two genes are wild type is normal, a normal mutation is a heterozygous mutation body, and two are mutations which are pure mutants.

### DETAILED DESCRIPTION OF THE INVENTION

These embodiments are illustrative of the invention and are not intended to limit the scope of the invention. The following experimental methods in the specific conditions are not indicated by methods are well known to researcher or the conditions recommended by the manufacturer. In addition, any methods and materials similar or equivalent to the contents described in the invention can be applied to all the. Implementation methods and materials described herein for demonstration purposes only.

The purpose of the invention is to provide a method for the effective realization of the transformation of the silk protein gene orientation, providing fibroin heavy chain mutation genomic resources. Namely, fibroin heavy chain gene targeted knockout, gets a series of fibroin heavy chain gene deletion, mutation or insertion mutants. In order to solve the above problems, the invention considers exogenous protein expression of the salivary glands silk proteins of endogenous protein purification process for subsequent major problems such as salivary glands bioreactor development faced by the use of the newly developed gene knockout technology, including a zinc-finger nucleases technology to the silk fibroin gene knockout, and mutants obtained genome sequencing and functional verification.

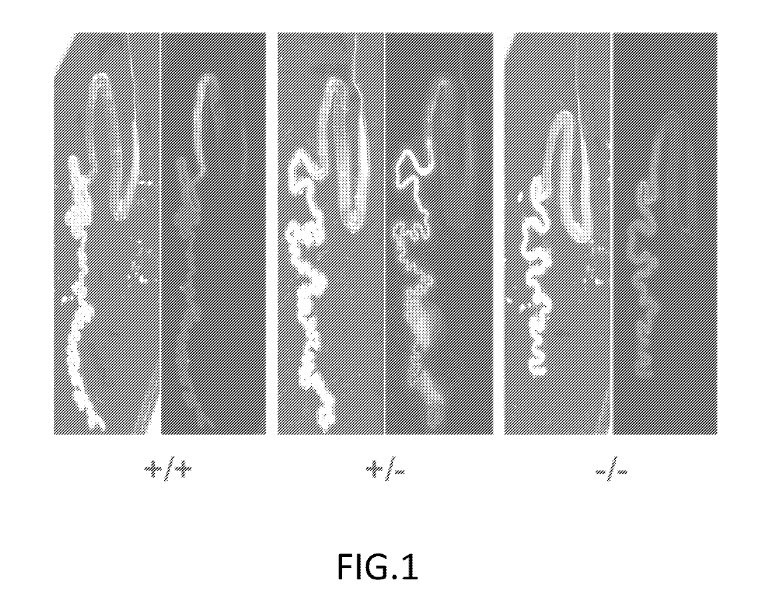

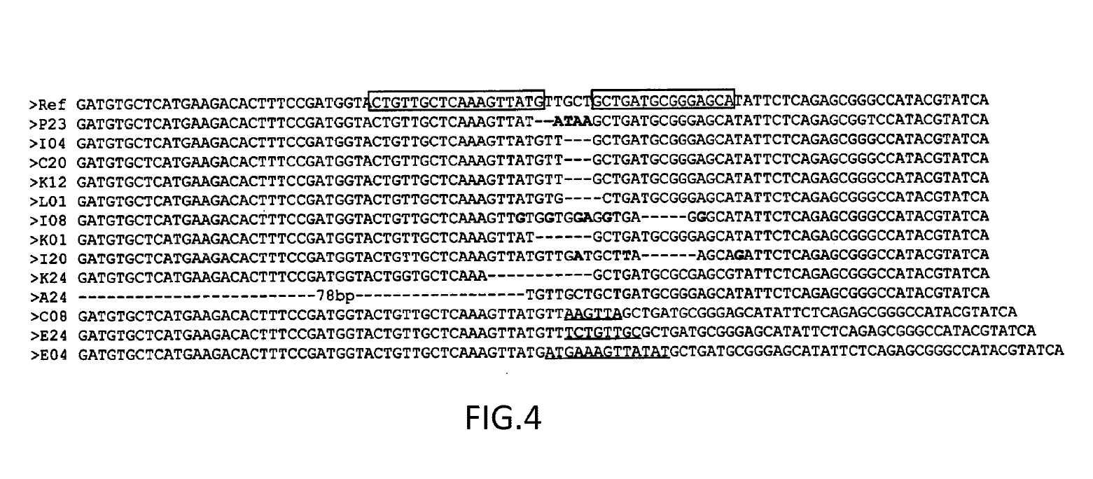

Referring to FIGS. 1 to 12, fibroin heavy chain gene mutant and preparation method of the invention comprises the following steps:

(1) downloading genomic sequences of heavy chain of fibroin, which analyzed mutant genes of the identified zinc-finger nucleases;

(2) regarding locus in the analysis of step (1), designing specific zinc-finger nucleases sequence;

(3) synthetic or from existing zinc-finger protein encoding library is amplified in step (2) in the design of zinc-finger nucleases a nucleic acid sequence;

(4) regarding step (3) a nucleic acid sequence is inserted into a prokaryotic expression vector containing a T7 or SP6 promoter and the recombinant vector;

(5) using the procedure of recombinant vector in step (4) obtained in vitro transcription to get coding of mRNA designed for zinc-finger nucleases in step (2);

(6) the silkmoth eggs diapause species through artificial incubation after treatment placed in 15° C., relative humidity of 75% of absolute dark environment incubation to hatching, non-diapause eggs of the silkworm Bombyx mori varieties placed at 25° C. 75% relative humidity of natural light environment incubation to hatching;

(7) daily collecting hatched silkworms by ants, placed in 25° C., relative humidity of 75% of the environment using natural light or artificial diet of mulberry leaves;

(8) transferring the silkworms to 25° C., relative humidity of 75% of a natural light environment for cocoon protection;

(9) collecting at the same time the emergence of male and female moths, at 25° C., low-light conditions after mating 4 hours and then dis-assembly, and the female silkmoths even put in sizing paper, produced in a dark environment eggs collected once per 0.5 hour, silkmoth eggs collected at 25° C. for protection;

(10) washing silkmoth eggs and the paper with tap water, soaked in distilled water to a paste expansion run (after about 2 minutes), then transferred the silkmoth eggs on a glass pasteurized by 75% alcohol, and in accordance with standard places silkmoth eggs to the right, neatly arranged silkmoth eggs in 35-37% formaldehyde vapor for sterilization 5 minutes;

(11) timing from collecting, 2 h-4 h after collection microinjection instrument will be used to mix mRNA obtained from step (5) in a molar ratio of 1:1, and then injecting it into silkmoth eggs which have been disinfected, and closing holes on egg shells by injecting a non-toxic glue;

(12) completing the injection of silkmoth eggs at 35-37% formaldehyde vapor for sterilization 5 min, placed at 25° C., relative humidity 85% of humidity conditions, carried incubation until hatching, raising silkworms from hatched ants, subjecting contemporary (G0) of moth to selfing or backcrossing to obtain G1 and further to obtain diapause silkmoth eggs;

(13) placing the G1 generation of silkmoth eggs obtained in step (12) via instant pickling treatment at 25° C., 75% relative humidity of natural light environments incubation to hatching;

(14) placing G1 ant silkworms obtained in step (13) at 25° C. of constant temperature, relative humidity of 75% of the environment using natural light conditions mulberry building a first cocoon, observing its silk and cocoon camp, camp or cocoon silk, and screening abnormal individual mutations;

(15) subjecting the mutations obtained in step (14) to selfing or backcrossing, seed and seed extract is obtained after the moth's genome, using silk fibroin heavy chain gene-specific primers for PCR amplification, sequencing, and further identifying mutants;

(16) sub-culturing the identified mutants obtained in step (15);

(17) based on the intended salivary glands protein expression of the amino acid sequence synthesized in vitro, or amplifying the coding sequence, and construction of target or gene transferring vector into the fibroin heavy chain gene by homologous recombination vector;

(18) taking silkmoth eggs obtained in step (16) as receptors, by using the steps (6)-(13) to store homologous recombination gene carrier transit carriers of step (17) injected into the step (16) to save the silkworm embryos, raising silkworms hatched ants, selfing or backcrossing contemporary (G0) of moth, getting diapause of the 01 generation of silkmoth eggs, G1 generation of silkmoth eggs through instant pickling treatment after the sixth day of embryonic development under a fluorescence microscope scanning transgenic individuals, transgenic individuals will get the normal feeding, subculture, and obtain the corresponding transgenic silkworm; and

(19) observing and analyzing salivary glands of transgenic cocoons obtained in step (18) in order to extract and purify proteins.

### Embodiment 1

### Sequence Analysis of Heavy Chain Gene of Fibroin

Downloaded from the NCBI database, fibroin heavy chain genomic sequence (number AF226688), the sequence of the structure shown in FIG. 1. Fibroin heavy chain gene (+1 to +16 788, where +1 indicates the transcription start site), including two long and 15750 bp are 67 bp exon and intron a long 971 bp, the first exon child including 25 bp untranslated region (+1 to +25) and 42 bp coding region (+26˜+67), the second exon containing the N-terminal non-repetitive region (+1039˜+1449), C terminal non-repetitive region (˜+16788+16396) and highly repetitive region (+1450˜+16395, gray box shown in FIG. 1). N-terminal amino acid sequence of the heavy chain fibroin contains a gene coding for 21 amino acid residues long signal peptide (underlined in FIG. 1 below).

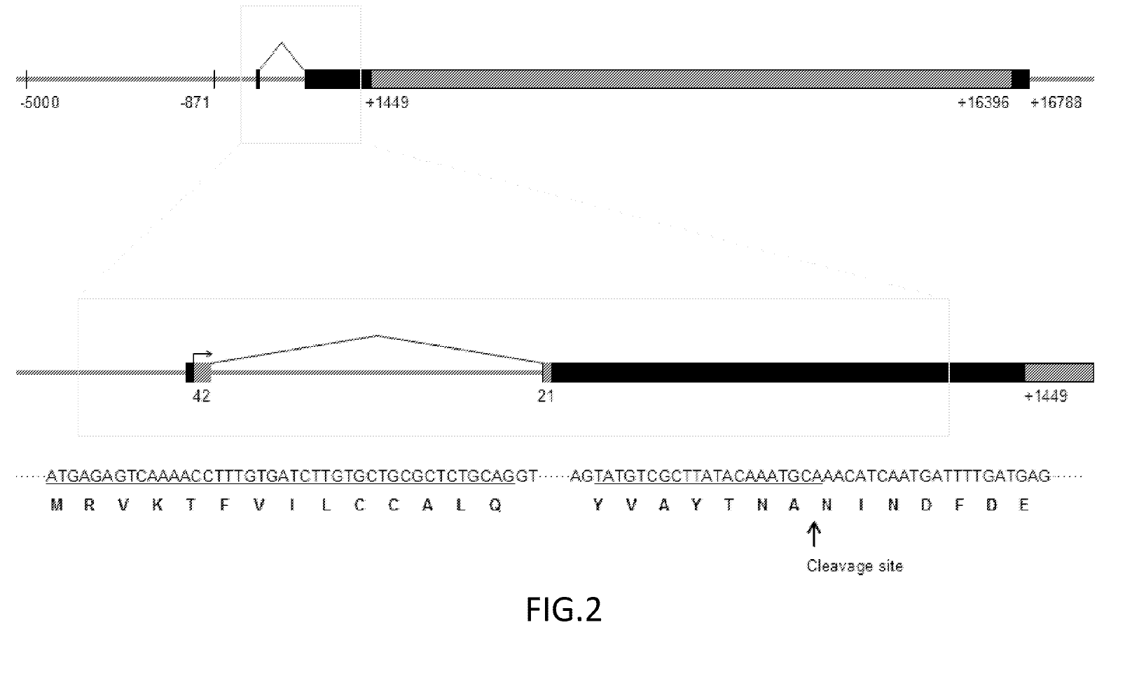

To select each can play a role in the silkworm strains of the identified zinc-finger nucleases, the N-terminal part of the sequence that we fibroin heavy chain gene (+1 to +1448) did SNP analysis, the results shown in FIG. 2 show. N-terminal part of the sequence of fibroin heavy chain gene (+289˜+1448) presence of 10 SNP loci in 29 strains and 11 wild silkmoth strains, respectively, +393, +465, +555, +556, +861, +862, +999, +1270 and +1390.

### Embodiment 2

fibroin heavy chain gene-specific zinc-finger nucleases design and synthesis sequence according to sequence features fibroin heavy chain gene and SNP in different silkworm strains, combined with the characteristics of zinc-finger proteins recognize DNA sequences, we choose CTGTTGCTCAAAGTTATGTTGCTGCTGATGCGGGAGCA zinc-finger nucleases recognition as the target point, the target point is located as SEQ ID NO: 1 showing the sequence˜+1362+1325 bits, and accordingly the design and synthesis of zinc-finger nucleases. Thus, fibroin heavy chain genes 1325˜1362 are taken as zinc-finger nucleases to identify targets.

### Embodiment 3

### Preparation of a Zinc-Finger Nucleases mRNA

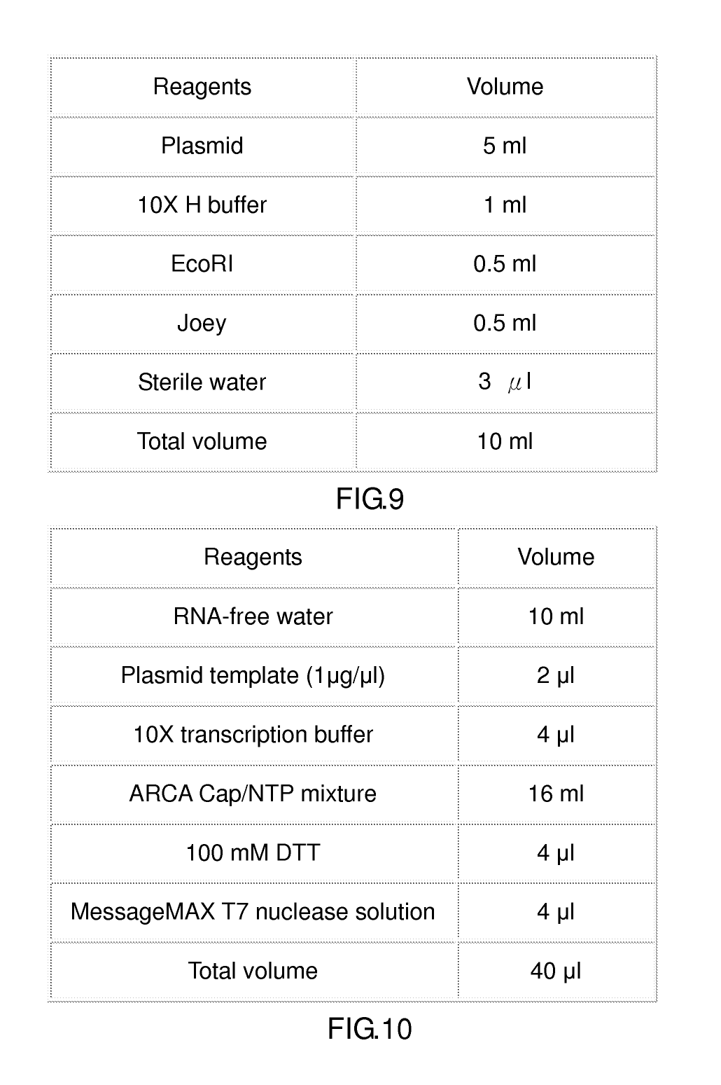

Synthetic or artificial zinc-finger protein from an existing library of amplified nucleic acid sequences of zinc-finger nucleases (e.g., SEQ ID NO: 119 and SEQ ID NO: 120) is digested with restriction endonucleases EcoRI and XhoI (purchased from TAKARA) after double digestion with the same restriction through prokaryotic expression vector pET28a ligation reaction, and positive clones are transformed into E. coli, the recombinant vector, a specific enzyme reaction system is shown in FIG. 9. The recombinant vector is digested with Xho I and in vitro transcription with MessageMax T7 mRNA using a vitro transcription kit (purchased from Epicentre), the reaction system is shown in FIG. 10.

The above reaction system is incubated for 30 minutes after adding the enzyme 1 μl DNA at 37° C., then incubation is continued for 15 minutes. The above reaction system tailing reaction tailed using Epicentre A-plus kit (purchased from Epicentre), the reaction system is shown in FIG. 11.

After the above-described system incubated at 37° C. 30 minutes and purified by MEGAClear kit (purchased from Ambion) at −80° C., it is stored for use.

### Embodiment 4

### Preparation of Microinjection Silkworm Embryos

Multivoltine silkworm breeds ‘N4’ approach: After at 25° C., 75% relative humidity environment incubation hatching, raising mulberry silkmoth eggs produced directly for microinjection.

Diapause silkworm variety ‘big build’ approach: the normal breeding silkmoth eggs obtained in the conventional artificial incubation is placed at 15° C. constant temperature and relative humidity of 75%, natural light environments incubation to hatching. Silkworm after hatching at 25° C. constant temperature, relative humidity of 75%, followed by natural light production environment for breeding silkmoth eggs for injection. After the emergence of moths, collected simultaneously male and female moth, after at 25° C., low-light conditions mating 4 h and then dis-assembly, and the female silkmoth even put on sizing paper for spawning.

### Embodiment 5

### Microinjection of Zinc-Finger Nucleases mRNA

Collecting silkworm paper and male and female moths simultaneously, at 25° C., low-light conditions after mating 4 h and then dis-assembly, and put on the female moth for spawning. And immediately after spawning, total concentration 400 ng/uL mori heavy chain gene targeting zinc-finger nucleases mRNA microinjection instrument (FemtoJet 5247 microinjection device, purchased from Eppendorf) is injected into silkmoth eggs among others, for each grain of silkmoth eggs injection volume is about 10 nL. Silkmoth eggs after injection with non-toxic glue to seal the injection hole, and by 35% of the formaldehyde vapor for sterilizing 5 min and later placed in 25° C., relative humidity of 85% high humidity incubation, the incubation of G0 generation of ants. Raising silkworms to artificial forage of moths.

### Embodiment 6

### Mutation Screening of Individuals after Microinjection

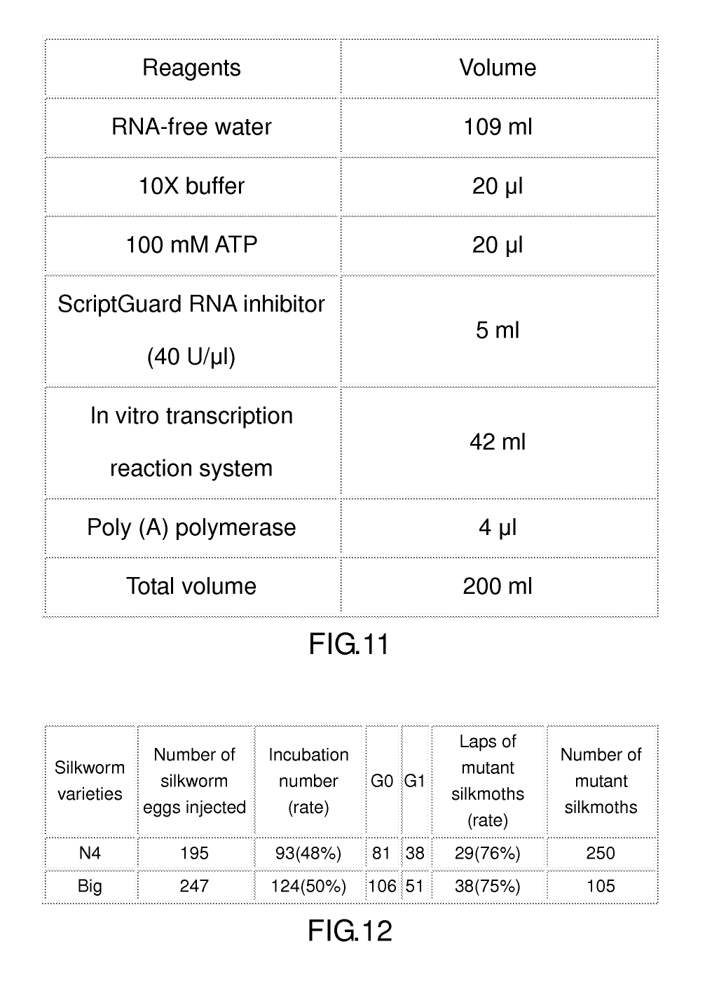

It uses multiple resistance varieties ‘N4’ and diapause varieties ‘big build’ as the raw material of silkmoth eggs injected 195 and 247, will hatch 93 and 124 G0 generation of artificial ants forage silkmoths reared to technology. 81 and 106, respectively, G0-generation moth obtained by selfing or backcrossing to get 38 and 51 on behalf of silkmoth eggs moth circle G1, the 38 and 51 moths circle incubation and raising alone to cocoon on the G1 generation for observation, respectively, in which 29 and 38 moths circle 250 and 105 are found in naked pupa or cocoon's thin ‘plastic cocoon’. Specific experiments statistical results are shown in FIG. 12.

### Embodiment 7

### Heritable Mutations Sequenced Individuals

From embodiment 6, 117 selected phenotypes are naked pupa or individual ‘glue cocoon’ mutations. Choose genomes. Select the genomic sequence near the fibroin heavy chain mutation design PCR primers and sequencing primers, PCR primers:

Fib-HF:
5′-tgatgaggactattttgggag-3′
Fib-HR:
5′-tagtgctgaaatcgctcgt-3′
Sequencing primers: Fib-HF:
5′-tgatgaggactattttgggag-3′

PCR reaction system as follows:

### Genomic DNA: 1.0 l

### EX Taq Buffer: 2.5 l

### Mg2+: 2.0 l

dNTP: 2.0 l

### Ex Taq: 0.15 μl

primers Fib-HF: 0.5 μl
primer Fib-HF: 0.5 l
ddH2O: up to 25 mu L

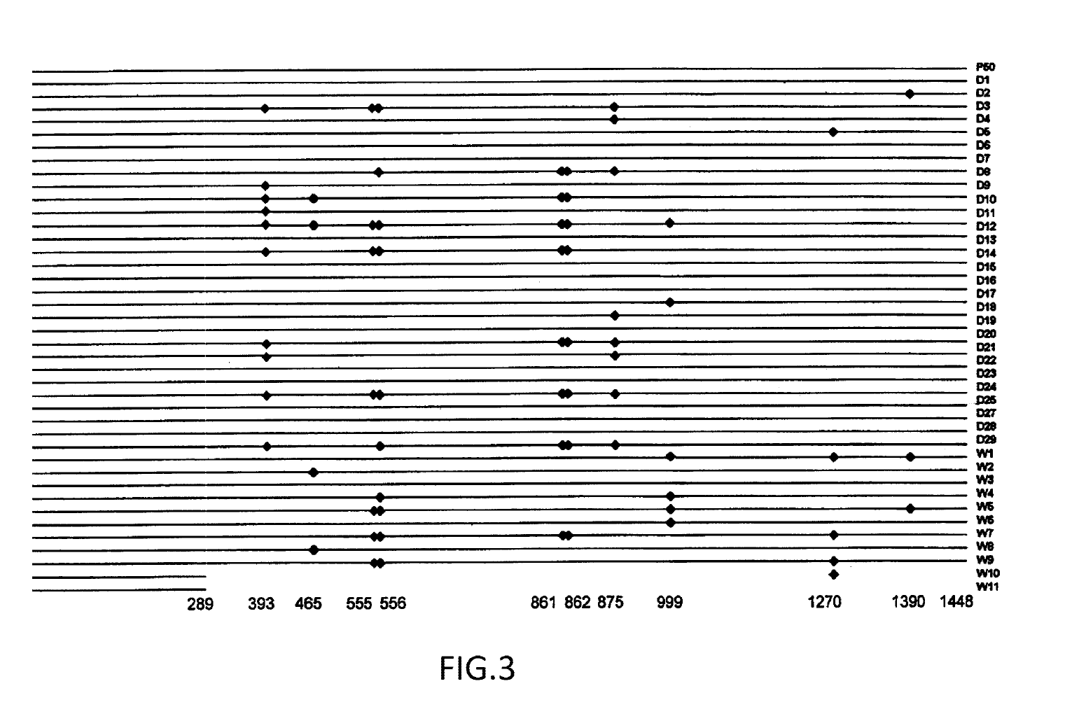

PCR products are electrophoresis, purified for sequencing reaction. The sequencing results show that for the selected 117 mutants, mutated genes of the identified zinc-finger nucleases, such as the specific sequence SEQ ID NO:2-118. The variation of these mutations includes insertion, deletion, and the small fragment, as shown in the partial results of FIG. 3.

### Embodiment 8

### Individual Genomic Mutant Phenotypes Observed and Anatomical Observations

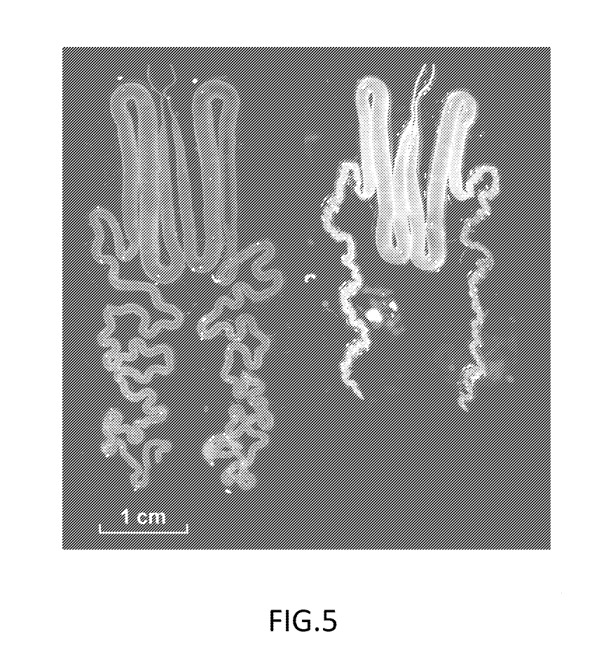

Taking strains of wild silkmoths and strains of 117 mutant silkmoths, three cocoons of either are taken to remove salivary glands by conventional anatomical for observation under the microscope, the results are shown in FIG. 5. Salivary glands of the mutant silkmoths are significantly less than that of the wild silkmoths. Further, posterior salivary glands of the mutant silkmoths appear to have dysplasia.

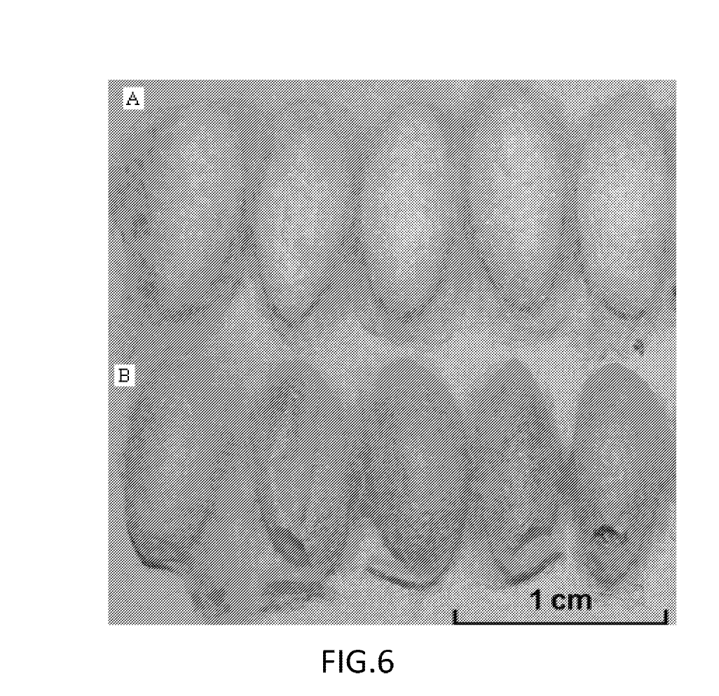

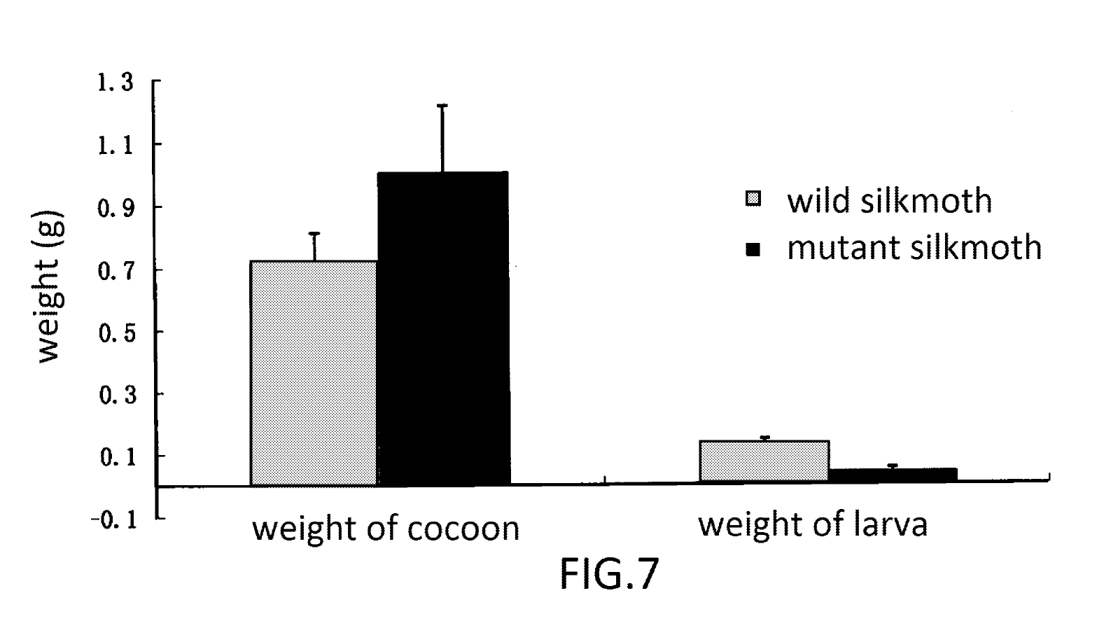

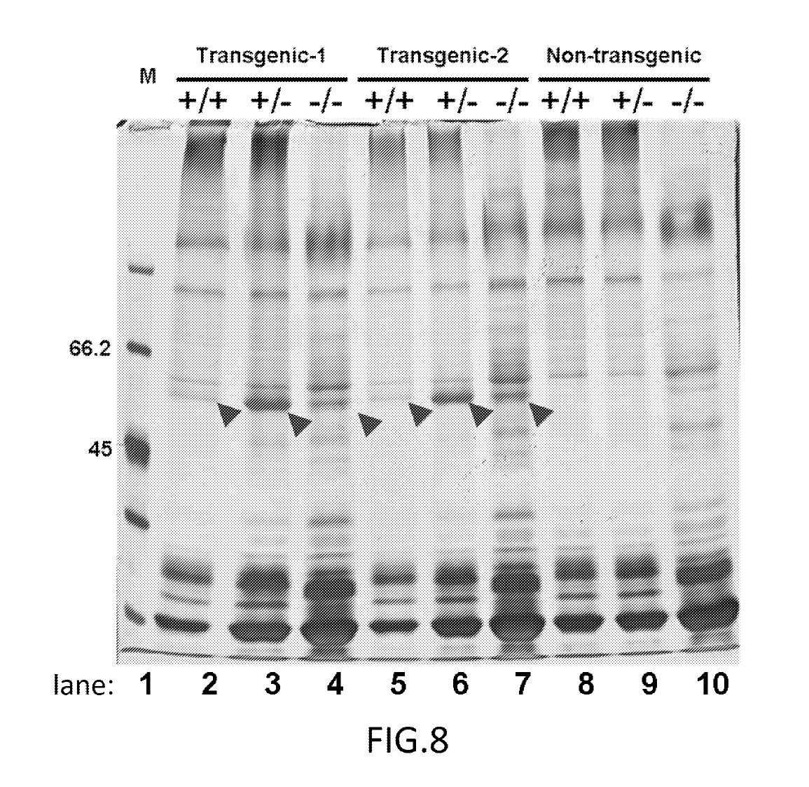

Taking strains of wild silkmoths and strains of 117 mutant silkmoths, 50 cocoons of either are taken to remove salivary glands by conventional anatomical for observation under the microscope, the results are shown in FIG. 6. Skins of the mutant silkmoths are thinner than that of the wild silkmoths. Further, 25 larvae and 25 cocoons of each of wild silkmoths and mutant silkmoths are taken for measuring their weights for biological statistical analysis, the results are shown in FIG. 7. Averagely, weights of larvae of the mutant silkmoths are greater than that of the wild silkmoths. Averagely, weights of cocoons of the mutant silkmoths are less that that of the wild silkmoths. Protein electrophoresis analysis is conducted on both strains of the wild silkmoths and the mutant silkmoths and results show that the mutant strains do not contain mori silk fibroin heavy chain protein, only the presence of a large number of sericin, see lane 10 of FIG. 8. Applications of fibroin heavy chain mutations are described in the preparation of endogenous proteins.

### Embodiment 9

### Exogenous Expression of Green Fluorescent Protein Fusion Mutants in the Salivary Glands

With a plasmid containing a green fluorescent protein gene as a template to amplify the green fluorescent protein coding sequence, and with the heavy chain fibroin non-repetitive region of the gene fusion constructs the transgenic vector (see construction methods and processes Aichun Zhao, etc., Transgenic Research, DOI 10.1007/s11248-009-9295-7). Use the strains of saved mutant silkmoths (mutant sequence as SEQ. ID NO: 11) as the acceptor material, under normal feeding conditions silkmoth eggs are obtained and the silkmoth eggs are placed in artificial incubation at 15° C., relative humidity of 75%, an absolutely dark environment incubation to hatching. At 25° C. constant temperature and relative humidity of 75% of a raising environment, cocoons are protected. After the emergence of moths, collecting simultaneously male and female moth, at 25° C., under low light conditions after mating 4 h and then dis-assembly, and the female silkmoth even put in sizing paper, in a dark environment, spawning, each interval of 0.5 h silkmoth eggs are collected once and collected silkmoth eggs are placed in 25° C. environment for protection. After spawning 3 h with Eppendorf microinjection instrument, the 10-15 nL, the total concentration of the constructed gene transfer 400 ng/uL 150 carriers injected into silkmoth eggs, using non-toxic glue to seal the injection hole, and by 35% Formaldehyde vapor for sterilization 5 min later, placed in 25° C., relative humidity of 85% high humidity incubation, the incubation of 69 G0 generation of ants mulberry silkmoth collection for raising and selfing or backcrossing, obtaining 11 laps of G1 generation of silkmoth eggs by using Olympus electric macroscopic fluorescence microscopy, screening 41 laps G1 generation of silkmoth eggs and getting a positive moth circles, and finally 11 transgenic silkworms are obtained. Salivary glands of transgenic silkworms and cocoons are observed and analyzed, the results are shown in FIG. 8. Green fluorescent protein content in salivary glands and cocoons of transgenic silkworms, which is taken as receptors in heterozygous and homozygous mutant silkmoths is significantly greater than that of the transgenic silkworms which is taken as the receptors of the wild silkmoths. Similarly, strains of 116 other mutant silkmoths (such as a mutant sequence SEQ. ID NO: 2˜10, SEQ. ID NO: 12˜118) as samples, green fluorescent protein content in salivary glands and cocoons of transgenic silkworms, which is taken as receptors in heterozygous and homozygous mutant silkmoths is significantly greater than that of the transgenic silkworms which is taken as the receptors of the wild silkmoths. Applications of the fibroin heavy chain mutation are described in the preparation of exogenous proteins.

While the invention has been described in terms of preferred embodiments, those skilled in the art will recognize that the invention can be practiced with modifications within the spirit and scope of the appended claims.

What is claimed is:

1. A mutation of a heavy chain of fibroin of silk emitted by Bombyx mori comprising:
genes 1325 to 1362 of a heavy chain of fibroin of silk emitted by Bombyx mori mutated as mutated genes of a target of identified zinc-finger nucleases.

2. The mutation of a heavy chain of fibroin of silk emitted by Bombyx mori of claim 1, wherein the mutated genes of a target of identified zinc-finger nucleases are any ones of SEQ. ID NO: 2-118.

3. The mutation of a heavy chain of fibroin of silk emitted by Bombyx mori of claim 2, wherein the mutated genes of a target of identified zinc-finger nucleases are any ones of SEQ. ID NO: 2-14.

4. The mutation of a heavy chain of fibroin of silk emitted by Bombyx mori of claim 2, wherein the mutated genes of a target of identified zinc-finger nucleases are SEQ. ID NO: 11.

5. The mutation of a heavy chain of fibroin of silk emitted by Bombyx mori of claim 1, wherein the mutated genes of a target of identified zinc-finger nucleases are described in the preparation of endogenous proteins.

6. The mutation of a heavy chain of fibroin of silk emitted by Bombyx mori of claim 1, wherein the mutated genes of a target of identified zinc-finger nucleases are described in the preparation of exogenous proteins.

7. A method of mutation of a heavy chain of fibroin of silk emitted by Bombyx mori, comprising the steps of:
inserting a zinc-finger nucleases sequence containing SEQ. ID NO: 119 and SEQ. ID NO: 120 into a prokaryotic expression vector of a promoter containing T7: ′-taatacgactcactataggg-3′ or SP6: 5′-atttaggtgacactatag-3 to create a recombinant vector;
transcribing in vitro of the recombinant vector to obtain an mRNA of coded zinc-finger nucleases;
taking the mRNA of coded zinc-finger nucleases as genes 1325 to 1362 of a heavy chain of fibroin of silk emitted by Bombyx mori mutated as mutated genes of a target of identified zinc-finger nucleases and indicated by SEQ. ID NO: 1; and
obtaining a mutation of a heavy chain of fibroin of silk emitted by Bombyx mori.

8. The method of mutation of a heavy chain of fibroin of silk emitted by Bombyx mori of claim 7, wherein the mutated genes of a target of identified zinc-finger nucleases are described in the preparation of sericin.


SEQUENCE LISTING (8 sequences)

SEQ ID NO: 1 - DNA, 20 bp, Bombyx mori
taatacgactcactataggg

SEQ ID NO: 2 - DNA, 18 bp, Artificial Sequence
  Primer
atttaggtgacactatag

SEQ ID NO: 3 - DNA, 88 bp, Bombyx mori
atgagagtcaaaacctttgtgatcttgtgctgcgctctgcaggtagtatgtcgcttatacaaatgcaaacatcaatgattttgatgag

SEQ ID NO: 4 - DNA, 1277 bp, Bombyx mori
gatgtgctcatgaagacactttccgatggtactgttgctcaaagttatgttgctgctgatgcgggagcatattctcagagcgggccatacgtatcagatgtgctcatgaagacactttccgatggtactgttgctcaaagttatataagctgatgcgggagcatattctcagagcggtccatacgtatcagatgtgctcatgaagacactttccgatggtactgttgctcaaagttatgttgctgatgcgggagcatattctcagagcgggtccatacgtatcagatgtgctcatgaagacactttccgatggtactgttgctcaaagttatgttgctgatgcgggagcatattctcagagcgggtccatacgtatcagatgtgctcatgaagacactttccgatggtactgttgctcaaagttatgttgctgatgcgggagcatattctcagagcgggtccatacgtatcagatgtgctcatgaagacactttccgatggtactgttgctcaaagttatgttctgatgcgggagcatattctcagagcgggtccatacgtatcagatgtgctcatgaagacactttccgatggtactgttgctcaaagttgtggtggaggtgagggcatattctcagagcgggtccatacgtatcagatgtgctcatgaagacactttccgatggtactgttgctcaaagttatgctgatgcgggagcatattctcagagcgggtccatacgtatcagatgtgctcatgaagacactttccgatggtactgttgctcaaagttatgttgatgcttaagcagattctcagagcgggtccatacgtatcagatgtgctcatgaagacactttccgatggtactgttgctcaaagctgatgcgcgagcgtattctcagagcgggccatacgtatcatgttgctgctgatgcgcgagcgtattctcagagcgggccatacgtatcagatgtgctcatgaagacactttccgatggtactgttgctcaaagttatgttaagttagctgatgcgggagcatattctcagagcgggccatacgtatcagatgtgctcatgaagacactttccgatggtactgttgctcaaagttatgtttctgttgcgctgatgcgggagcatattctcagagcgggccatacgtatcagatgtgctcatgaagacactttccgatggtactgttgctcaaagttatgatgaaagttatatgctgatgcgggagcatattctcagagcgggccatacgtatca

SEQ ID NO: 5 - DNA, 38 bp, Artificial Sequence
  Primer
ctgttgctcaaagttatgttgctgctgatgcgggagca

SEQ ID NO: 6 - DNA, 21 bp, Artificial Sequence
  Primer
tgatgaggactattttgggag

SEQ ID NO: 7 - DNA, 19 bp, Artificial Sequence
  Primer
tagtgctgaaatcgctcgt

SEQ ID NO: 8 - DNA, 21 bp, Artificial Sequence
  Primer
tgatgaggactattttgggag